(19) United States
(12) Patent Application Publication
(10) Pub. No.: US 2020/0171216 A1
(43) Pub. Date: Jun. 4, 2020

(54) ADHESION PREVENTION MATERIAL

(71) Applicant: KAWASUMI LABORATORIES, INC., Oit (JP)

(72) Inventors: Tomokazu MUKAI, Oita (JP); Noriaki SHIRAHAMA, Oita (JP)

(21) Appl. No.: 16/640,364

(22) PCT Filed: Aug. 27, 2018
(86) PCT No.: PCT/JP2018/031569

§ 371 Date: Feb. 20, 2020

(30) Foreign Application Priority Data
Aug. 28, 2017 (JP) 2017-163678

(51) Int. Cl. A61L 31/16; A61L 31/04; A61L 31/14; A61L 31/10; A61K 31/375

(52) U.S. Cl. CPC A61L 31/16; A61L 31/042; A61L 2300/428; A61L 31/10; A61K 31/375; A61L 31/148

(57) ABSTRACT

An objective of the present invention is to provide an adhesion prevention material having improved barrier performance. The objective is achieved by a solid or semi-solid adhesion prevention material comprising a bioabsorbable material, said adhesion prevention material containing, as an active ingredient, a cytostatic factor demonstrating cell proliferation inhibiting effect.

### TECHNICAL FIELD

The present invention relates to an adhesion prevention material.

### BACKGROUND ART

Conventionally, an adhesion prevention material is known that reduces the adhesion of living tissue that can occur due to surgery or trauma (for example, see Patent Document 1). This adhesion prevention material provides an important barrier function that physically shields and separates a wound site from other biological tissues until the wound site tissue is repaired or healed.

### PRIOR ART DOCUMENT

### Patent Document

Patent Document 1: Japanese Patent No. 5686297

### SUMMARY OF THE INVENTION

### Technical Problem

Also, an improvement in the adhesion prevention effect may be achieved by changing the physical properties and form of the adhesion prevention material. However, for example, there are also limitations in the physical properties and form of the adhesion prevention material when the ease of handling of the adhesion prevention material is considered in the context of endoscopic surgery, which uses a laparoscope or the like. Consequently, the physical barrier function that originates from the physical properties and form of the adhesion prevention material may not be sufficient.

In particular, if an adhesion prevention material is composed of a bioabsorbable material, the barrier function is not sufficiently demonstrated if the absorption is too fast. On the other hand, if the absorption rate is reduced too much, there is a concern that foreign matter may indefinitely remain inside the body even after the wound site is repaired. For this reason, an adhesion prevention material having a suitable bioabsorbability, while also having an improved barrier performance, is desired.

Therefore, the present invention has been made to solve such a problem, and has an object of providing an adhesion prevention material with improved barrier performance.

### Solution to Problem

In order to solve the problem described above, an aspect of the present invention is a solid or semi-solid adhesion prevention material including: a cytostatic factor having a cell proliferation inhibiting effect as an active ingredient.

### Advantageous Effect of the Invention

According to the present invention, an adhesion prevention material having an improved barrier function can be provided.

### BRIEF DESCRIPTION OF THE DRAWINGS

FIG. 1 is a diagram schematically illustrating an embodiment of an adhesion prevention material according to the present invention.

FIG. 2 is a diagram illustrating the results of a cell proliferation inhibition evaluation test.

### DESCRIPTION OF THE EMBODIMENT

Hereinafter, an embodiment of the present invention will be described with reference to the drawings.

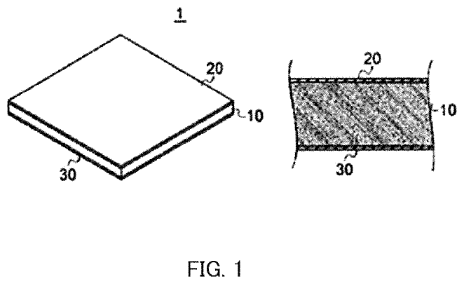

FIG. 1 is a diagram schematically illustrating an embodiment of an adhesion prevention material according to the present invention. FIG. 1(a) is a perspective view of an adhesion prevention material 1, and FIG. 1(b) is a partially enlarged cross-sectional view thereof. In FIG. 1(a) and FIG. 1(b), a base body layer 10, a first coating layer 20, and a second coating layer 30 are schematically illustrated with the thickness exaggerated.

The adhesion prevention material 1 of the present embodiment is a solid or semi-solid adhesion prevention material 1 containing a bioabsorbable material, and includes a cytostatic factor having a cell proliferation inhibiting effect as an active ingredient.

Examples of the solid form of the adhesion prevention material 1 include a film form, a sheet form, and a mesh form. Examples of the semi-solid form include a gel form, such as a colloid form.

A base body constituting the adhesion prevention material 1 is substantially composed of a water-soluble polymer (A) and a polyaliphatic ester (B) (the details will be described later). Specifically, in the case of a laminated structure in which the coating layers 20 and 30 are formed covering the surface (one or both sides) of the base body layer 10, for example, the base body layer 10 is substantially constituted by only the water-soluble polymer (A). Furthermore, for example, the coating layers 20 and 30, which are relatively thin compared to the base body layer 10, are substantially constituted by only the polyaliphatic ester (B). Here, while the term “substantially” allows for the inclusion of trace amounts of impurities that unavoidably become mixed during production and the like, it signifies that other components have not been intentionally added.

Furthermore, the base body constituting the adhesion prevention material 1 may have a single-layer structure. In this case, the base body is referred to as a “base body layer”. In addition, in the case of a single-layer structure, for example, the base body can be substantially composed of a two-component composition (A/B) consisting of the water-soluble polymer (A) and the polyaliphatic ester (B). The base body constituting the adhesion prevention material 1 may also be substantially composed of only the water-soluble polymer (single component).

Moreover, there is no clear distinction between a film form and a sheet form. However, in the present invention, a film form refers to a thickness of less than 200 [μm], and a sheet form refers to a thickness of 200 [μm] or more. Also, a mesh form refers to a fibrous structure composed of a composite in which a base body substantially composed of the solid water-soluble polymer and the aliphatic ester has been formed into a rod shape. Here, the basis weight is in a range of 0.800 to 830 [g/m²].

The semi-solid adhesion prevention material 1 has a single-layer structure or a laminated structure. If a barrier function is to be demonstrated, the viscosity at 37 [° C.] is preferably about 100 to 1,000,000 [Pa·s].

As illustrated in FIG. 1(a) and FIG. 1(b), the adhesion prevention material 1 includes a film form base body layer 10, a first coating layer 20 disposed on the surface on one side of the base body layer 10 (the upper side in the drawing), and a second coating layer 30 disposed on the surface on the other side of the base body layer 10 (the lower side in the drawing).

### <Basic Configuration of Base Body>

The base body of the present invention has a single-layer structure of a two-component composition substantially composed of the water-soluble polymer (A) and the polyaliphatic ester (B), or a laminated structure which includes the base body layer 10, which is constituted by the water-soluble polymer (A), and coating layers (the first coating layer 20 and the second coating layer 30), which are constituted by the polyaliphatic ester (B). Because the polyaliphatic ester (B) component does not decompose as quickly as the water-soluble polymer (A) when placed in a living body, the layered form (film form or sheet form) is retained for a considerably longer period of time. On the other hand, the water-soluble polymer (A) component is mixed with, or is in contact with, the polyaliphatic ester (B), which serves as a shape-retaining component. Therefore, for example, dissolution does not occur as rapidly as for the case of a single-component layer, and gradual elution takes place from the sections of the surface of the base body which are in contact with biological fluid.

As described above, the water-soluble polymer (A) component is gradually eluted from the substrate (sustained release). It is expected that the sustained release also causes the additive (cytostatic factor) included in the adhesion prevention material 1 to also be gradually eluted in the same manner.

Furthermore, the elution amount (elution rate) of the water-soluble polymer (A) increases as the proportion of the water-soluble polymer (A) constituting the base body is increased. However, if the proportion of the water-soluble polymer (A) is made too large, the proportion of the polyaliphatic ester (B) decreases. Therefore, the ability of the film form to be retained is reduced. On the other hand, if the proportion of the polyaliphatic ester (B) is increased, the form of the base body, such as the film form, is stably retained for a long period of time. However, the elution rate of the water-soluble polymer (A) is reduced.

From this perspective, in the present invention, the weight proportion ω (=A/B) of the water-soluble polymer (A) and the polyaliphatic ester (B) that constitute the base body is 1˜99/99˜1. Furthermore, ω (=A/B) is more preferably 20˜80/80˜20, and ω (=A/B) is even more preferably 30˜70/70˜30. If the proportion of the water-soluble polymer (A) is much smaller than this, the elution rate becomes low. Also, the sustained release rate of the additive (cytostatic factor) or the like included in the adhesion prevention material 1 becomes very low. On the other hand, if the amount of the polyaliphatic ester (B) becomes much greater than this, despite an improvement in the shape retention properties, the elution rate of the water-soluble polymer (A) and the sustained release rate of the additive become too low.

### <Water-Soluble Polymer>

Firstly, the water-soluble polymer (A) will be described.

Preferably used as the water-soluble polymer (A) are, for example, polysaccharides, proteins, and synthetic polymers.

Preferably used as the polysaccharides are, for example, animal and plant storage polysaccharides such as starch, amylose, amylopectin, glycogen, glucomannan, dextrin, glucan, and fructan; animal structural polysaccharides such as cellulose, pectin, and chitin; polysaccharides derived from seaweed such as carrageenan and agarose; microbial polysaccharides such as pullulan; plant gum polysaccharides such as locust bean gum and guar gum; glycosaminoglycans such as heparin, hyaluronic acid, chondroitin sulfate, heparan sulfate, dermatan sulfate, and keratan sulfate; and other derivatives of these polysaccharides.

Preferably used as the protein are, for example, gelatin, casein, and collagen.

Preferably used as the synthetic polymer are, for example, polyvinyl alcohol, polyvinyl alcohol derivatives, polyacrylic acid-based water-soluble polymers, polyacrylamide, polyacrylamide derivatives, polyethylene oxide, polyethylene oxide derivatives, polyvinyl pyrrolidone, polyvinyl pyrrolidone derivatives, polyamide polymers, polyalkylene oxide polymers, polyether glycol polymers, and maleic anhydride copolymers.

Among the exemplified water-soluble polymers (A), pullulan can be particularly preferably used from the perspective of increasing the flexibility of the adhesion prevention material 1 as a whole.

### <Polyaliphatic Ester>

Next, the polyaliphatic ester (B) will be described.

Preferably used as the polyaliphatic ester (B) are, for example, poly(lactides); poly(glycolides); poly(lactide-ε-glycolides); poly(lactic acids); poly(glycolic acids); Poly(lactic acid-ε-glycolic acids) polycaprolactones; polyesteramides; polyanhydrides; polyorthoesters; polycyanoacrylates; polyetheresters; poly(dioxanones); poly(alkylene alkylates); copolymers of polyethylene glycol and polyorthoester, and other copolymers; and polymer alloys.

In particular, due to the excellent biocompatibility, it is preferable to use at least one of poly(lactic acids), poly(glycol acids), polycaprolactones, or their copolymers. Particularly preferable is a lactic acid/glycolic acid/ε-caprolactone ternary copolymer (LA/GA/ε-CLT) having a molecular weight of about 20,000 to 300,000.

### <Base Body Layer>

The thickness of the base body layer 10 is set to, for example, about 1 to 5,000 [μm]. The base body layer 10 includes, in addition to those that configure the adhesion prevention material 1 having a laminated structure, the base body of an adhesion prevention material having a single-layer structure.

In the case of a film form adhesion prevention material, the thickness of the base body layer 10 is less than 200 [μm], preferably 1 to 150 [μm], more preferably 10 to 100 [μm], and most preferably 30 to 80 [μm].

In the case of a sheet form adhesion prevention material, the thickness of the base body layer 10 is 200 [μm] or more, preferably 200 to 5,000 [μm], more preferably 300 to 3,000 [μm], even more preferably 500 to 2,000 [μm], and most preferably 800 to 1,000 [μm].

In addition to measurement by direct contact by a vernier caliper or microgauge, the thickness of the base body layer 10 can be measured, for example, by using an appropriate device such as an infrared film thickness gauge, a capacitance thickness gauge, or a laser displacement sensor.

### <Coating Layer>

The coating layers (the first coating layer 20 and the second coating layer 30) have an optical thickness which is set to 50 to 7,000 [nm], preferably 100 to 5,000 [nm], and more preferably about 1,000 to 3,000 [nm] when measured at a wavelength of 380 to 900 [nm] using a spectroscopic ellipsometer. Here, for example, even when the optical thickness of the coating layers is set to about 50 to 100 [nm], as described below, because the addition of a cytostatic factor or the like is an aspect of the present invention, a sufficient adhesion prevention performance can be demonstrated.

In addition, when the adhesion prevention material 1 has a laminated structure formed of a base body layer 10, which is substantially composed of the water-soluble polymer (A), and the coating layers (the first coating layer 20 and the second coating layer 30) formed thereon, which are substantially composed of the polyaliphatic ester (B), the function of the coating layers is basically thought to enable control of the elution rate of the water-soluble polymer (A) in the base body layer 10, and the sustained release rate of the additive.

For this reason, if the coating layer is too thick, there is a concern that the elution rate of the water-soluble polymer (A) of the base body layer 10 and the sustained release rate of the additive will become too low. However, from the perspective of maintaining the strength (retaining the form) of the base body layer 10 and the adhesion prevention material 1 as a whole, it is desirable for the coating layers to have a sufficient thickness so that they are strong enough to prevent easy breakage. That is to say, in bipedal animals such as monkeys and apes, or in bipedal animals such as humans, there is a concern that an increase in the intraabnominal pressure or movement of organs such as the stomach or intestines may cause a breakage when the base body layer 10 and the coating layers (the first coating layer 20 and the second coating layer 30) of the adhesion prevention material 1 are configured as a film form or sheet form film. Therefore, when priority is given to ensuring that the mechanical strength of the coating layers is sufficient, it is considered desirable for the optical thickness of the coating layers to be, for example, 800 to 7,000 [nm], preferably 900 to 5,000 [nm], and more preferably about 1,000 to 3,000 [nm].

Furthermore, as described above, it is possible to provide basic control over the elution rate of the water-soluble polymer (A) (and the sustained release rate of the additive) by changing the weight ratio ω (=A/B) of the water-soluble polymer (A) and the polyaliphatic ester (B) that constitute the base body layer. However, it is also possible to more precisely control the elution rate of the water-soluble polymer (A) and the sustained release rate of the additive by additionally forming the coating layers (the first coating layer 20 and the second coating layer 30) and controlling their thickness within the ranges described above.

Therefore, when various conditions are considered, such as the intraabnominal pressure in which the adhesion prevention material 1 is placed, the force environment, the type of target organ, and the required sustained release period, the thickness of the coating layers is determined depending on whether the elution rate of the water-soluble polymer (A) and the sustained release rate of the additive is prioritized, the shape retention is prioritized, or both are prioritized.

### <Cytostatic Factor>

Next, the cytostatic factor will be described.

When the adhesion prevention material 1 is applied to a wound site inside a living body, the cytostatic factor has a cell proliferation inhibiting effect with respect to the cells in contact with the adhesion prevention material 1.

Furthermore, when the adhesion prevention material 1 has a laminated structure, the cytostatic factor may be included in the base body layer 10, included in the coating layers (the first coating layer 20 and the second coating layer 30), or included in both. If the cytostatic factor is included in the base body layer 10, sustained release of the cytostatic factor occurs as the water-soluble polymer (A) of the base body layer 10 is eluted. Moreover, if the cytostatic factor is included in the coating layers (the first coating layer 20 and the second coating layer 30), the cell proliferation inhibiting effect with respect to the cells in contact with the adhesion prevention material 1 is demonstrated even in the initial states of application of the adhesion prevention material 1. That is to say, because the eluted water-soluble polymer (A), such as pullulan, forms a viscous solution, it is expected that the solution will remain in the same position while also containing the cytostatic factor. As a result, because a viscous pullulan solution or the like which includes the cytostatic factor stably covers the wound site, it is thought that the wound site becomes protected by a synergistic effect between the cytostatic factor and pullulan or the like, enabling an adhesion prevention effect to be demonstrated.

The thickness of the coating layers (the first coating layer 20 and the second coating layer 30) is significantly thinner than the thickness of the base body layer 10. Therefore, because the total amount of the cytostatic factor included in the coating layers cannot be made very large, it is considered to provide an effect which is secondary to that of the cytostatic factor included in the base body layer 10.

Examples of the cytostatic factor include an acid, an anticancer agent, a cell inhibitor, an anti-inflammatory agent, a steroid, an antibacterial agent, and an antibiotic agent. The adhesion prevention material 1 includes at least one of these.

The acid may be an organic acid or an inorganic acid, and examples include ascorbic acid (and ascorbic acid derivatives) and hydrochloric acid.

For example, the L-form of ascorbic acid (L-ascorbate), which known as vitamin C, may be preferably used. Examples of ascorbic acid derivatives include calcium ascorbate, sodium ascorbate, sodium phosphate-L-ascorbate, magnesium phosphate-L-ascorbate, glucoside ascorbate, and ethyl ascorbic acid.

Known anticancer agents such as cisplatin may be appropriately used.

Known cell inhibitors such as zinc diethyldithiocarbamate (ZDEC) may be appropriately used.

Known anti-inflammatory agents such as acetylsalicylic acid and acetaminophen may be appropriately used.

Known steroids such as dexamethasone may be appropriately used.

Known antibacterial agents such as norfloxacin may be appropriately used.

Known antibiotic agents such as cefoperazone sodium (third-generation cephem) may be appropriately used.

Examples of cytostatic factors that may be used include vitamin E; carotenoids such as α-carotene, ß-carotene, γ-carotene, lycopene, and xanthophyll, which are fat-soluble pigments of plants; and plant-derived antioxidant substances (SOD substances) including polyphenols such as flavonoid, catechin, tannin, anthocyanin, isoflavone, and quercetin, which are included in the flowers, leaves, bark, and stems and the like of plants.

### EXAMPLES

### Examples 1 to 3

The adhesion prevention material was prepared by adding a predetermined amount of L-ascorbic acid (manufactured by Wako Pure Chemical Industries, Ltd.) to the material of the base body layer, and forming a first coating layer and a second coating layer made of the same material on both sides of the base body layer, which was molded as a film form. The samples of Examples 1 to 3 were obtained after the pH was adjusted to a predetermined range.

### (Formation of Base Body Layer)

Pullulan, which is a water-soluble polymer, was used as the material of the base body layer. After a predetermined amount of ascorbic acid was added thereto, a 100 [mm]×120 [mm]×50 [μm] thick film substantially composed of pullulan was formed by the casting method, thereby forming an ascorbic acid-containing base body layer.

### (Formation of Coating Layer)

A toluene solution (hereinafter referred to as a coating solution) of polylactic acid-polyglycolic acid-poly-ε-caprolactone, which is a polyaliphatic ester, was prepared having a concentration adjusted to a predetermined concentration. Then, the base body layer prepared above was immersed in the coating solution, and coating layers substantially composed of a polylactic acid-polyglycolic acid-poly-ε-cap rolactone ternary copolymer were formed on both surfaces of the base body layer.

After dipping, the samples (test pieces) corresponding to Examples 1 to 3 were obtained by drying for approximately 30 minutes to 1 hour at room temperature.

The optical thickness of the coating layers was measured using a spectroscopic ellipsometer (“alpha-SE (US registered trademark)” manufactured by J. A. Woollam Japan). The measurement wavelength was 380 to 900 [nm].

### (Concentration of Ascorbic Acid)

The thickness of the coating layers substantially composed of polylactic acid-polyglycolic acid-poly-ε-caprolactone was fixed to 300 [nm], and the concentration of ascorbic acid Cm (w/w) % added to the base body layer was varied. The pH was measured 24 hours after immersion in bovine plasma.

In Example 1, an addition concentration of ascorbic acid Cm of 2.0 (w/w) % resulted in a pH of 7.0. In Example 2, a Cm of 4.0 (w/w) % resulted in a pH of 7.0. In Example 3, a Cm of 8.0 (w/w) % resulted in a pH of 6.0.

### Comparative Example 1

In Comparative Example 1, ascorbic acid was not added to the base body layer. Otherwise, the adhesion prevention material was prepared in the same manner as in Examples 1 to 3.

The thickness of the coating was 300 [nm]. The pH was 7.3 after immersion for 24 hours in bovine plasma.

### [Evaluation Test of Adhesion Prevention Performance]

When the adhesion prevention performance was evaluated, the samples (test pieces) corresponding to Examples 1 to 3 and Comparative Example 1 were applied to the inside of the abdominal cavity of a pig, and the adhesion prevention performance was evaluated by observing and scoring the extent of adhesion.

### (Production of Adhesion Model)

The abdomen of a pig was opened by a 15 [cm] midline abdominal incision under general anesthesia, and the small intestine was exposed outside the wound. Next, a certain area (approximately 1×5 [cm]) of the exposed small intestine was abraded with a file until punctate bleeding occurred.

Air exposure was carried out for exactly 10 minutes after punctate bleeding occurred. Then, the small intestine was returned to the abdominal cavity, and the adhesion prevention materials corresponding to the test pieces described above were applied directly beneath the incised section. The abdominal wall was continuously sutured and closed in two layers using an absorbable suture (2-0), and an adhesion model was prepared.

### (Observation of Extent of Adhesion, Calculation of Adhesion Score)

Fourteen days after the preparation of the adhesion model, the pig was exsanguinated and sacrificed under general anesthesia. Then, the abdomen was opened, and the adhesion directly beneath the midline was visually observed to determine an adhesion occurrence probability. Because the present evaluation relates to the lower abdomen, adhesion to the liver located in the upper abdomen was ignored.

The test results are shown in Table 1.

TABLE 1

| | Adhesion occurrence rate σ |
|---|---|
| | (Number of pigs with adhesion |
| | observed/total number of pigs) |
| Comparative | 66% (2/3) |
| Example 1 | |
| Example 1 | 20% (2/5) |
| (Cm = 2.0%) | |
| Example 2 | 0% (0/5) |
| (Cm = 4.0%) | |
| Example 3 | 0% (0/5) |
| (Cm = 8.0%) | |

### (Consideration)

In Examples 1-3, in which ascorbic acid was added to the base body layer, it was confirmed that the adhesion occurrence rate σ was significantly reduced when compared with Comparative Example 1, in which ascorbic acid was not added. Furthermore, it was confirmed that when the addition concentration of ascorbic acid Cm was increased from 2.0 (w/w) % (Cm 4.0 (w/w) % and Cm 8.0 (w/w) %), the adhesion occurrence rate σ further reduced from 20% to 0%.

As a result, the adhesion prevention material becomes a physical barrier between wound sites, and cell proliferation is inhibited at the wound sites by the cytostatic factor (ascorbic acid), which is gradually released with the elution of the pullulan (water-soluble polymer (A)) in the base body layer. It is thought that a synergistic effect between the cytostatic factor and pullulan or the like provides an appropriate bioabsorption performance, while also enabling the barrier performance to be further improved.

### Examples 11-18

The samples of Examples 11 to 18 were obtained using an adhesion prevention material in which a first coating layer and a second coating layer containing a predetermined additive and made of the same material were disposed on both sides of the base body layer, which was molded as a film form.

### (Formation of Base Body Layer)

Pullulan, which is a water-soluble polymer, was used as the material of the base body layer. After a predetermined additive was added and adjusted to a predetermined concentration, a 100 [mm]×120 [mm]×50 [μm] thick film substantially composed of pullulan was formed by the casting method, thereby forming an additive-containing base body layer.

### (Formation of Coating Layer)

A toluene solution (hereinafter referred to as a coating solution) of polylactic acid-polyglycolic acid-poly-ε-caprolactone, which is a polyaliphatic ester, was prepared. Then, the base body layer prepared above was immersed in the coating solution, and coating layers substantially composed of a polylactic acid-polyglycolic acid-poly-ε-caprolactone ternary copolymer was formed on both surfaces of the base body layer.

After dipping, the samples (test pieces) corresponding to Examples 11 to 18 were obtained by drying for approximately 30 minutes to 1 hour at room temperature.

The optical thickness of the coating layers were measured using a spectroscopic ellipsometer (“alpha-SE (US registered trademark)” manufactured by J. A. Woollam Japan). The measurement wavelength was 380 to 900 [nm].

### (Additive)

The test piece of Example 11 was obtained by adding 6.13 [mg] of ascorbic acid (manufactured by Wako Pure Chemical Industries, Ltd.) per 2×2 [cm] of the sample.

Similarly, the test piece of Example 12 was obtained by adding 3.07 [mg] of hydrochloric acid (manufactured by Wako Pure Chemical Industries, Ltd.) per 2×2 [cm] of the sample.

Similarly, the test piece of Example 13 was obtained by adding 0.31 [mg] of cisplatin (manufactured by Nippon Kayaku Co., Ltd.) per 2×2 [cm] of the sample.

Similarly, the test piece of Example 14 was obtained by adding 3.07 [mg] of zinc diethyldithiocarbamate (manufactured by Tokyo Chemical Industry Co., Ltd.) per 2×2 [cm] of the sample.

Similarly, the test piece of Example 15 was obtained by adding 3.07 [mg] of acetylsalicylic acid (manufactured by Wako Pure Chemical Industries, Ltd.) per 2×2 [cm] of the sample.

Similarly, the test piece of Example 16 was obtained by adding 3.07 [mg] of acetaminophen (manufactured by Toronto Research Chemicals Inc.) per 2×2 [cm] of the sample.

Similarly, the test piece of Example 17 was obtained by adding 0.31 [mg] of dexamethasone (manufactured by Toronto Research Chemicals Inc.) per 2×2 [cm] of the sample.

Similarly, the test piece of Example 18 was obtained by adding 3.07 [mg] of norfloxacin (manufactured by Wako Pure Chemical Industries, Ltd.) per 2×2 [cm] of the sample.

### Comparative Examples 11 to 16

In Comparative Examples 11 to 16, the type of additive added to the base body layer is different to Examples 11 to 18. Otherwise, the adhesion prevention material was prepared in the same manner as in Examples 11 to 18.

### (Additive)

The test piece of Comparative Example 11 was obtained by adding 3.07 [mg] of citric acid (manufactured by Wako Pure Chemical Industries, Ltd.) per 2×2 [cm] of the sample.

Similarly, the test piece of Comparative Example 12 was obtained by adding 3.07 [mg] of sodium hydroxide (manufactured by Wako Pure Chemical Industries, Ltd.) per 2×2 [cm] of the sample.

Similarly, the test piece of Comparative Example 13 was obtained by adding 3.07 [mg] of sodium hydrogen carbonate (manufactured by Wako Pure Chemical Industries, Ltd.) per 2×2 [cm] of the sample.

Similarly, the test piece of Comparative Example 14 was obtained by adding 3.07 [mg] of sodium citrate (manufactured by Wako Pure Chemical Industries, Ltd.) per 2×2 [cm] of the sample.

Similarly, the test piece of Comparative Example 15 was obtained by adding 3.07 [mg] of salicylic acid (manufactured by Wako Pure Chemical Industries, Ltd.) per 2×2 [cm] of the sample.

Similarly, the test piece of Comparative Example 16 was obtained by adding 7.67 [mg] of sodium chloride (manufactured by Wako Pure Chemical Industries, Ltd.) per 2×2 [cm] of the sample.

### [Evaluation Test of Cell Proliferation Inhibition]

When the cell proliferation inhibition performance was evaluated, the samples (test pieces) corresponding to Examples 11 to 18 and Comparative Examples 11 to 16 were applied to a culture medium containing a culture of mouse L929 fibroblasts, and the cell proliferation inhibition performance was evaluated by observing (by visual inspection) the extent of cell proliferation.

### (Sample Preparation)

The mouse L929 fibroblasts were cultured in a minimum essential medium (MEM medium). The number of cells was adjusted to 105 cells per 1 [ml], and 5 [ml] of the adjusted solution was seeded in a 25 [cm²] petri dish. Then, the culture was allowed to stand for 24 hours at 37° C. under a 5% carbon dioxide environment.

After this time, a 2×2 [cm] test piece was applied to the cultured petri dish, and the culture was allowed to stand for 5 days at 37° C. under a 5% carbon dioxide environment.

Then, a Giemsa staining solution was added dropwise to the cultured petri dish to stain the cells.

### (Observation and Evaluation of Extent of Cell Proliferation)

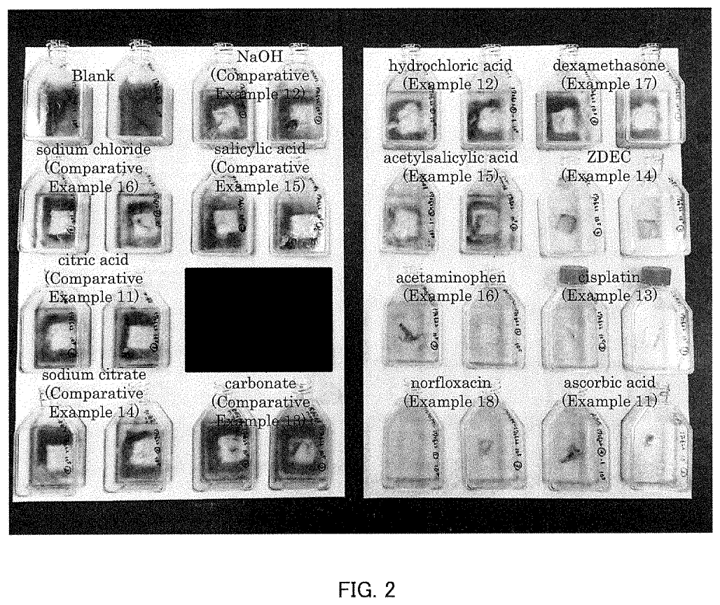

The test results are illustrated in FIG. 2.

The sample without an applied test piece is designated as a blank (upper left), and the cell proliferation inhibition performance of each test piece was confirmed by visual inspection using the degree of staining (extent of cell proliferation) of the blank as a reference.

### (Consideration)

As illustrated in FIG. 2, it was clearly confirmed that the adhesion prevention materials with added ascorbic acid, hydrochloric acid, cisplatin, zinc diethyldithiocarbamate (ZDEC), acetylsalicylic acid, acetaminophen, dexamethasone, or norfloxacin as an additive (Examples 11-18) had a cell proliferation inhibiting property.

That is to say, similarly to the adhesion prevention material with added ascorbic acid (Example 11), it is thought that the adhesion prevention materials with added hydrochloric acid, cisplatin, zinc diethyldithiocarbamate (ZDEC), acetylsalicylic acid, acetaminophen, dexamethasone, or norfloxacin (Examples 12-18) inhibit cell proliferation and are capable of improving barrier performance. Furthermore, if a cytostatic factor is added to the base body layer, it is thought that a synergistic effect between the cytostatic factor and pullulan (water-soluble polymer (A)) or the like of the base body layer provides an appropriate bioabsorption performance, while also enabling the barrier performance to be further improved.

In the cell proliferation inhibition evaluation test, for example, tests were not performed using antibiotic agents such as cefoperazone sodium as the additive. However, because antibiotic agents inhibit the proliferation and function of cells, they are thought to be effective as a cytostatic factor included in the adhesion prevention material.

Furthermore, by similarly including a cytostatic factor in a semi-solid adhesion prevention material, the adhesion prevention material forms a physical barrier between wound sites. Also, cell proliferation at the wound sites is suppressed by the cytostatic factor, which is gradually released with the elution of the base body. Therefore, it is thought that the barrier performance can be further improved.

As described above, the adhesion prevention material 1 of the present embodiment is a solid or semi-solid adhesion prevention material 1, and includes a cytostatic factor having a cell proliferation inhibiting effect as an active ingredient. Consequently, adhesion to wound sites can be prevented as a result of the physical properties and form of the adhesion prevention material 1, and further, cell proliferation can be inhibited at the wound site by means of the cytostatic factor. Therefore, an adhesion prevention material 1 having an improved barrier performance can be provided.

In particular, because the adhesion prevention material 1 is made of a bioabsorbable material, an appropriate bioabsorbability is provided, while also enabling the barrier performance to be further improved due to the inclusion of the cytostatic factor.

The present invention is not limited to the embodiment described above, and various improvements and design changes may be made within a scope not departing from the spirit of the present invention.

Furthermore, the adhesion prevention material may be configured to exhibit a sol-gel transition phenomenon in response to temperature. Specific configurations of such adhesion prevention materials are disclosed, for example, in Japanese Unexamined Patent Application Publication No. 2014-221736 and Japanese Unexamined Patent Application Publication No. 2016-189894. Therefore, a detailed description is omitted.

In addition, the embodiment disclosed above is to be considered illustrative in all aspects, and is not intended to be restrictive. The scope of the present invention is defined by the scope of claims, and not by the description above. It is intended that all modifications that fall within the meaning and scope equivalent to the scope of claims are to be included.

Priority is claimed on Japanese Patent Application No. 2017-163678 filed Aug. 28, 2017, and the contents described in the claims, the specification and the drawings of the application are herein incorporated by reference.

### REFERENCE SIGNS LIST

1 Adhesion Prevention Material
10 Base Body Layer
20 First Coating Layer
30 Second Coating Layer

1. A solid or semi-solid adhesion prevention material comprising:
a cytostatic factor having a cell proliferation inhibiting effect as an active ingredient.

2. The adhesion prevention material according to claim 1, comprising
a bioabsorbable material.

3. The adhesion prevention material according to claim 1, wherein
the cytostatic factor includes at least one of an acid, an anticancer agent, a cell inhibitor, an anti-inflammatory agent, a steroid, an antibacterial agent, and an antibiotic agent.
